(19) United States
(12) Patent Application Publication
(10) Pub. No.: US 2018/0246025 A1
(43) Pub. Date: Aug. 30, 2018

(54) Device and Method for Determining Viscosity

(71) Applicant: Bayerische Motoren Werke Aktiengesellschaft, Muenchen (DE)

(72) Inventor: Johannes ESCHL, Groebenzell (DE)

(21) Appl. No.: 15/963,453

(22) Filed: Apr. 26, 2018

Related U.S. Application Data
(63) Continuation of application No. PCT/EP2016/069370, filed on Aug. 16, 2016.

(30) Foreign Application Priority Data
Oct. 27, 2015 (DE) 10 2015 220 966.0

(51) Int. Cl. G01N 11/08; G01N 33/44

(52) U.S. Cl. CPC G01N 11/08; G01N 33/442

(57) ABSTRACT

A device for determining the viscosity of reaction resins is provided. The device includes a sample storage unit with at least two storage containers and respectively a dosing unit, at least one mixing device, a high pressure pump, a temperature-control unit, and at least one capillary with an inlet and an outlet. A pressure measuring device is arranged at least on each capillary's outlet and on the inlet of the first capillary.

### CROSS REFERENCE TO RELATED APPLICATIONS

This application is a continuation of PCT International Application No. PCT/EP2016/069370, filed Aug. 16, 2016, which claims priority under 35 U.S.C. §119 from German Patent Application No. 10 2015 220 966.0, filed Oct. 27, 2015, the entire disclosures of which are herein expressly incorporated by reference.

### BACKGROUND AND SUMMARY OF THE INVENTION

The present invention relates to a device for determining the viscosity of reactive resins, and to a method for producing such a device. The invention furthermore also relates to an easily implementable method for determining viscosity.

Determinations of viscosity are used for evaluating the flow characteristics of substances and reaction mixtures. Different measurement methods are conventional for this purpose. In the measurement methods, a distinction is made between those which measure a force (for example a moment) at a specific shear rate, and those which determine the viscosity from the time which a sample to be analyzed requires in order to flow through a capillary with a defined volume. The former measurement method is unsuitable for determining the viscosity of reactive resins since the sample preparation takes too long and the resins are already undergoing curing during the carrying out of the actual measurement, this leading to a distortion of the measurement results. A disadvantage of the latter measurement method is that the dosing of reactive substances, and the control of the temperature thereof, are difficult and laborious. Also, in the measurement of reactive resins, the capillary is generally not reused.

Proceeding from said prior art, it is an object of the present invention to provide a device for determining the viscosity of reactive resins, which device allows precise dosing of the substances to be reacted, quick temperature control and determination of viscosity with low error potential, and makes it possible for a statement to be made regarding the temporal processing window of reactive resins. It is also an object of the present invention to specify a method for producing a device for determining the viscosity of reactive resins, which method is easily implementable and has recourse to commercially available components. A further object is also to specify a method for determining the viscosity of reactive resins, by way of which method the viscosity of reactive resins can be detected precisely without great sample preparation effort.

This and other objects are achieved by a device for determining the viscosity of reactive resins in accordance with embodiments of the invention. The individual components of the device according to embodiments of the invention are described below. The device thus includes a sample storage unit having at least two storage containers and in each case one dosing unit. Any container which is inert with respect to the substance to be stored is suitable as storage container. Here, the dosing unit allows precise separation and delivery of a defined quantity of substance.

Since the device according to an embodiment of the invention for determining viscosity is a device for determining the viscosity of reactive resins, at least two storage containers are provided, a first one for storing a curable resin and a second one for storing a curing agent. The resin and the curing agent are not restricted in terms of their detail. Thermosetting resins, for example epoxy resins or polyurethane resins, wherein amines, isocyanates and the like are suitable as the curing agent, may be mentioned by way of example. Further storage containers, for example for storing, delivering and dosing reaction additives, solvents, separating agents and the like, may be provided.

The device according to an embodiment of the invention further includes at least one mixing device. The mixing device is designed to mix at least the curable resin and the curing agent with one another. The mixing device may in this case be integrated into a dosing device.

In order to achieve adequate flow of the reaction mixture of the reactive resin, the device also includes a high-pressure pump. By way of the high-pressure pump, the reaction mixture, that is to say the mixture of curable resin and curing agent, is brought to a specific pressure, as a result of which a predefined volumetric flow rate can be set.

The high-pressure pump advantageously generates a maximum pressure of 100 to 130 bar in the mixture of curable resin and curing agent. This ensures leak-tightness of the system, which is reduced with increasingly higher pressures such that reaction mixture components can flow out, for example, between a piston and a cylinder of the high-pressure pump, as a result of which the high-pressure pump volumetric flow rate is damaged irreversibly.

Furthermore, the temperature of the reaction mixture is controlled by way of a temperature-control unit. Temperature control may include both increasing the temperature of the reaction mixture and lowering the temperature thereof, as is conventional during the curing reaction, for example.

The viscosity is subsequently determined during the flow of the reaction mixture through at least one capillary. Here, a capillary includes an inlet into which the reaction mixture is admitted and an outlet through which the reaction mixture exits the capillary after flowing through the latter. A pressure measurement device, for example a pressure sensor, is present at least at each outlet of a capillary. If only one capillary is present in the device, said capillary also includes at its inlet a pressure measurement device. If several capillaries are connected in series, then at least the first capillary includes at its inlet a pressure measurement device.

The capillary is not restricted in terms of its detail. Preferably, its length and its diameter are selected such that the throughflow time through the capillary corresponds approximately to the injection time which is required to deliver, for example in a resin injection process, a specific, intended quantity of reactive resin.

By determining the pressure difference between two pressure measurement devices, the viscosity measurement can be determined according to the Hagen-Poiseuille law:

$${V}^{′} = \frac{π \cdot {r}^{4} \cdot Δ p}{8 \cdot η \cdot l}$$

where V′ is the predefined volumetric flow rate in m³/s through the capillary, η is the viscosity, r is the radius of the capillary, Δp is the pressure difference between two pressure measurement devices, and l is the length of the capillary.

The device according to an embodiment of the invention additionally has yet another advantage. It allows the processing time of a reactive resin to be determined via the determined viscosity. The processing time is in this case that time during which the reaction mixture is still sufficiently free-flowing and before the resin has been crosslinked, by way of reaction with the curing agent, such that delivery at the desired speed is no longer possible. The determination of the processing time is important for example for reactive resins which are used in injection devices for producing fiber composite components and the like. If the processing time is known, it is possible for the dosing to be set correspondingly such that no defects or inhomogeneities occur in the cured resin.

The device according to an embodiment of the invention is thus able to be used in a versatile manner for determining the viscosity of reactive resins and allows quick, precise and cost-effective determination of the viscosity. Due to the unique dosing system, the possibility exists for mixing multiple substances and also for flushing the device by way of a suitable solvent after carrying out the viscosity measurement. This allows the device to be reused without replacing one of its components. The structure of the device according to the embodiment of the invention is compact and thus is also suitable as a mobile measurement device.

According to an advantageous refinement, the dosing unit includes a pump, and in particular a double-piston pump. This allows substances to be dosed without vortices, which prevents measurement errors.

Likewise according to an embodiment of the invention, a method for producing a device for determining the viscosity of reactive resins is also described. Here, it is pointed out that the method according to the invention is suitable for producing the device according to the invention discussed above. The method includes the step of i) providing an HPLC (high performance liquid chromatography) system, which includes a sample storage unit having at least two storage containers and in each case one dosing unit, at least one mixing device, a high-pressure pump, a temperature-control unit and at least one separating column. The method further includes the step of ii) replacing the separating column by at least one capillary having an inlet and an outlet, wherein a pressure measurement device is arranged at least at each outlet of a capillary and at the inlet of the first capillary.

Accordingly, a conventional HPLC system may be used as a starting basis. Only the separating column is replaced by a capillary, and pressure measurement devices are, where necessary, provided. Consequently, the production of a device for determining the viscosity of reactive resins, which device works in an efficient, quick and cost-effective manner, is able to be produced in a very simple way using conventional equipment. Moreover, the method is realizable without great effort.

Furthermore according to an embodiment of the invention, a method for determining the viscosity of reactive resins is also described. The method includes the following steps: i) dosing and delivering a curable resin from a first storage container of a sample storage unit by way of a first dosing unit, and ii) dosing and delivering a curing agent from a second storage container of the sample storage unit by way of a second dosing unit. The curable resin and the curing agent are in this case selected such that a curing reaction, in general a crosslinking reaction, can be initiated between the substances. This takes place after step iii), the mixing of the curable resin and the curing agent in a mixing device. Then, in step iv), a predefined volumetric flow rate of the mixture of curable resin and curing agent is set by way of a high-pressure pump, v) the temperature of the mixture of curable resin and curing agent is controlled, and vi) the reaction mixture, that is to say the mixture of curable resin and curing agent, is delivered through at least one capillary having an inlet and an outlet, wherein a pressure measurement device is arranged at least at each outlet of a capillary and at the inlet of the first capillary. Subsequently, vii) a pressure difference which arises between two pressure measurement devices at the given volumetric flow rate of the mixture of curing agent and curable resin is determined. From this, as described above for the device according to the invention for determining the viscosity of reactive resins, it is possible for the viscosity to be determined according to the Hagen-Poiseuille law. The method is realizable in a time-saving manner and allows precise determination of the viscosity of reactive resins.

For further details, reference is additionally made to the above statements regarding the device according to embodiments of the invention for determining the viscosity of reactive resins.

Preferably, after the throughflow of the mixture of curable resin and curing agent, the capillary is freed of residues by dosing and delivering from a third storage container through the capillary a solvent for the cured resin.

Other objects, advantages and novel features of the present invention will become apparent from the following detailed description of one or more preferred embodiments when considered in conjunction with the accompanying drawings.

### BRIEF DESCRIPTION OF THE DRAWING

FIG. 1 is a schematic illustration of a device for determining the viscosity of reactive resins according to an advantageous configuration of the invention.

### DETAILED DESCRIPTION OF THE DRAWING

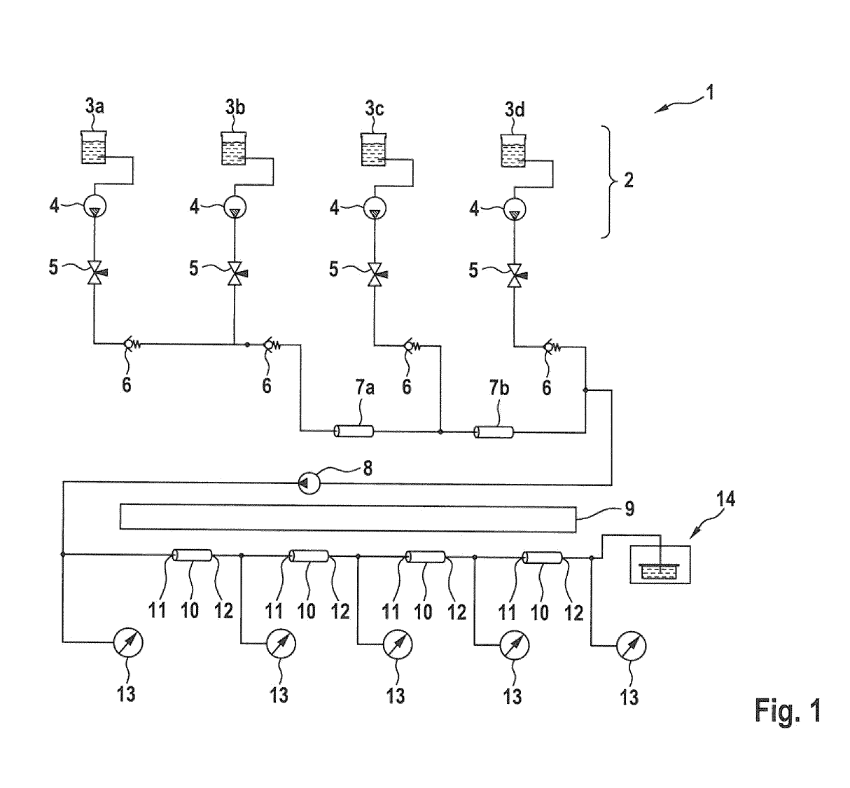

The present invention will be discussed in detail on the basis of an exemplary embodiment. To this end, only those aspects of the invention which are essential to the invention are illustrated in FIG. 1. All other aspects have been omitted for the sake of clarity.

The device 1 according to an embodiment of the invention includes a sample storage unit 2 having four storage containers 3a, 3b, 3c, 3d. The storage containers 3a, 3b, 3c, 3d are each adjoined by a dosing unit 4 by way of which a corresponding quantity of a stored substance can be dosed and delivered. Needle valves 5 and backflow prevention valves 6 prevent the substances from flowing back.

The substances delivered from the storage containers 3b and 3c and from the storage containers 3b/3c and 3d are mixed with one another in mixing devices 7a and 7b, respectively.

The sample storage unit 2 and the mixing devices 7a, 7b are situated in the so-called low-pressure region of the device 1. This means that here substantially no pressure increase has taken place, and the substances and mixtures are generally at normal pressure.

The low-pressure region is adjoined by a high-pressure region. The latter region is reached after a high-pressure pump 8 has been passed through.

By way of example, the device 1 contains four capillaries 10 in the high-pressure region. Each capillary has an inlet 11 and an outlet 12. A pressure measurement device 13 is arranged at each outlet 12 of each capillary 10. A pressure measurement device 13 is likewise also arranged at the inlet 11 of the first capillary 10. A pressure measurement device 13 is therefore present in each case at the inlet 11 and at the outlet 12 of each capillary 10. This allows a pressure difference between the inlet 11 of a capillary 10 under consideration and the outlet 12 of said capillary 10 to be measured. It is likewise possible to determine a pressure difference over multiple capillaries 10.

The device 1 further includes a temperature-control unit 9 by way of which all the capillaries 10 can be brought to a desired temperature.

Connected downstream of the capillaries 10 is a collecting container 14 in which substances delivered through the capillaries 10 are collected for subsequent disposal.

The functioning of the device 1 will be explained on the basis of the following example:
The storage containers 3a-3d are filled as follows:
Storage container 3a: Solvent for a reactive resin;
Storage container 3b: Curing agent;
Storage container 3c: Separating agent; and
Storage container 3d: Curable resin.

A predefined quantity of curing agent is dosed and delivered from the storage container 3b via the adjoining dosing unit 4. A predefined quantity of curable resin is dosed and delivered from the storage container 3d via the adjoining dosing unit 4. The curable resin and the curing agent are mixed with one another in the mixing device 7b and delivered in the line to the high-pressure pump 8.

A predefined volumetric flow rate of the mixture of curable resin and curing agent is set by way of the high-pressure pump 8.

After passing through the high-pressure pump 8, the mixture of curing agent and curable resin is delivered through the capillaries 10 at constant temperature, and this is set and maintained by the temperature-control unit 9. The pressure measurement devices 13 allow a pressure difference to be determined at an arbitrary location between the capillaries 10.

If a pressure difference which arises between two pressure measurement devices 13 at the given volumetric flow rate of the mixture of curing agent and curable resin is then determined, it is then possible from this for the viscosity of the reactive resin, that is to say of the mixture of curing agent and curable resin, to be determined via the Hagen-Poiseuille law.

Also, by way of tests and a comparison of the determined parameters, it is possible for the time which a reactive resin is still sufficiently free-flowing before it has cured completely to be determined. These findings are helpful for the process control in an injection process for the reactive resin under consideration, for example in an RTM process.

After the viscosity measurement has ended, it is possible for a solvent for the reactive resin to be delivered from the storage container 3a, for example, and pumped through the capillaries 10. This prevents blockage of the capillaries 10 with resin and allows repeated use of the capillaries 10.

It is equally also possible to supply a separating agent from the storage container 3c.

### LIST OF REFERENCE SIGNS

1 Device for determining the viscosity of reactive resins
2 Sample storage unit
3a Storage container
3b Storage container
3c Storage container
3d Storage container
4 Dosing unit
5 Needle valve
6 Backflow prevention valve
7a Mixing device
7b Mixing device
8 High-pressure pump
9 Temperature-control unit
10 Capillary
11 Inlet of the capillary
12 Outlet of the capillary
13 Pressure measurement device
14 Collecting container

The foregoing disclosure has been set forth merely to illustrate the invention and is not intended to be limiting. Since modifications of the disclosed embodiments incorporating the spirit and substance of the invention may occur to persons skilled in the art, the invention should be construed to include everything within the scope of the appended claims and equivalents thereof.

What is claimed is:

1. A device for determining a viscosity of reactive resins, comprising:
a sample storage unit including at least two storage containers and in each case one dosing unit;
at least one mixing device;
a high-pressure pump;
a temperature-control unit; and
at least one capillary having an inlet and an outlet, wherein
a pressure measurement device is arranged at least at each outlet of the at least one capillary and at an inlet of a first capillary.

2. The device according to claim 1, wherein the dosing unit includes a pump.

3. The device according to claim 2, wherein the pump is a double-piston pump.

4. A method for producing a device for determining a viscosity of reactive resins, the method comprising the acts of:
providing an HPLC system, wherein the HPLC system includes a sample storage unit having at least two storage containers and in each case one dosing unit, at least one mixing device, a high-pressure pump, a temperature-control unit, and at least one separating column;
replacing the separating column by at least one capillary having an inlet and an outlet; and
arranging a pressure measurement device at least at each outlet of the at least one capillary and at an inlet of a first capillary.

5. A method for determining a viscosity of reactive resins, the method comprising the acts of:
dosing and delivering a curable resin from a first storage container of a sample storage unit by way of a first dosing unit;
dosing and delivering a curing agent from a second storage container of the sample storage unit by way of a second dosing unit;
mixing the curable resin and the curing agent in a mixing device;
setting a predefined volumetric flow rate of the mixture of the curable resin and the curing agent by way of a high-pressure pump;
controlling a temperature of the mixture of the curable resin and the curing agent;
delivering the mixture of the curable resin and the curing agent through at least one capillary having an inlet and an outlet;
arranging a pressure measurement device at least at each outlet of the at least one capillary and at an inlet of a first capillary; and
determining a pressure difference which arises between two pressure measurement devices at a given volumetric flow rate of the mixture of the curing agent and the curable resin.

6. The method according to claim 5, the method further comprising the act of:
after the throughflow of the mixture of the curable resin and the curing agent, freeing the at least one capillary of residues by dosing and delivering from a third storage container through the at least one capillary a solvent for the cured resin.
